(19) United States
(12) Patent Application Publication
(10) Pub. No.: US 2005/0186232 A1
(43) Pub. Date: Aug. 25, 2005

(54) Hair-relaxing composition comprising tetramethylguanidine

(76) Inventors: Gerard Malle, Villiers S/Morin (FR); Damien Lavergne, Paris (FR)

Correspondence Address: Thomas L. Irving;FINNEGAN, HENDERSON, FARABOW,, GARRETT & DUNNER, L.L.P., 1300 I Street, N.W., Washington, DC, 20005-3315, US

(21) Appl. No.: 10/988,571

(22) Filed: Nov. 16, 2004

Related U.S. Application Data
(60) Provisional application No. 60/562,297, filed on Apr. 15, 2004.

(30) Foreign Application Priority Data
Nov. 18, 2003 (FR) 03 50849

(51) Int. Cl. A61K 7/075; A61K 7/08; A61K 7/09

(52) U.S. Cl. 424/401; 424/70.27; 424/70.2

(57) ABSTRACT

Disclosed herein is a ready-to-use cosmetic composition for relaxing keratin fibers, comprising at least one tetramethylguanidine as relaxing agent. Further disclosed herein are a kit comprising at least two compartments to be brought into contact to form the ready-to-use composition, and a process using this composition.

This application claims benefit of U.S. Provisional Application No. 60/562,297, filed Apr. 15, 2004.

The present disclosure relates to a ready-to-use cosmetic composition for relaxing keratin fibers, comprising at least one tetramethylguanidine as relaxing agent. The present disclosure also relates to a kit comprising at least two compartments to be brought into contact to form the ready-to-use composition, and to a process using this composition.

As used herein, the term “tetramethylguanidine” means 1,1,3,3-tetramethylguanidine (Registry Number [80-70-6]) and the organic and mineral salts thereof. The organic and mineral salts of tetramethylguanidine are known to one of ordinary skill in the art.

As used herein, the term “keratin fibers” means fibers of human or animal origin such as head hair, other body hairs, the eyelashes, wool, angora, cashmere and fur. Although the present disclosure is not limited to particular keratin fibers, in one embodiment, the keratin fibers are head hair.

Further as used herein, the term “relaxing” includes the relaxing, smoothing-out or decurling of Caucasian, Asiatic, North African or African hair.

In the present disclosure, the term “between x % and y %” means ranging from x % to y %, wherein the limits x % and y % are included.

Two techniques may be used to permanently reshape the hair. They are based on breaking the disulfide bonds present in keratin (cystine):
The first technique comprises, in a first step, opening the disulfide bonds by a composition comprising a reducing agent, and then, after having, for example, rinsed the hair, in reconstituting, in a second step, the disulfide bonds by applying to the hair, which has been placed under tension beforehand with curlers or the like, or shaped or smoothed out by other methods, an oxidizing composition also known as a fixing composition, so as to give the head of hair the desired shape. This technique makes it possible either to make the hair wavy or to relax it, decurl it or straighten it out.
The second technique comprises performing a lanthionization operation, using a composition comprising at least one base belonging to the hydroxide family, i.e., at least one hydroxide base. This can lead to replacement of the disulfide bonds (—CH₂—S—S—CH₂—) with lanthionine bonds (—CH₂—S—CH₂—). This lanthionization operation involves two consecutive chemical reactions:
   The first reaction includes a beta-elimination on the cystine caused by a hydroxide ion, leading to the breaking of this bond and the formation of dehydroalanine. 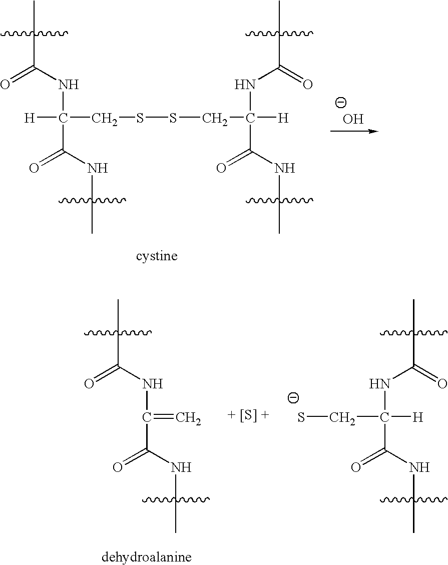
   The second reaction is a reaction of the dehydroalanine with a thiol group. Specifically, the double bond of the dehydroalanine formed is a reactive double bond. It can react with the thiol group of the cysteine residue that has been released to form a new bond, referred to as a lanthionine bridge, bond or residue.

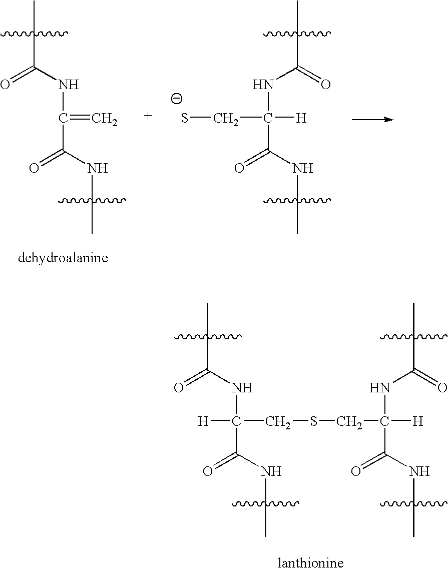

Different from the first technique that uses a reducing agent, this lanthionization technique does not require a fixing step, since the formation of the lanthionine bridges is irreversible. It may thus take place in a single step and makes it possible either to make the hair wavy or to relax it, decurl it or straighten it out. However, it is mainly used to relax naturally frizzy or curly hair.

For the first technique, the reducing compositions generally used for the first step of a permanent-waving or relaxing operation comprise at least one reducing agent chosen from thiols, sulfites and bisulfites. These agents are generally used in essentially aqueous medium at concentrations of between 0.5 and 1 M to obtain good opening of the disulfide bonds. Among the thiols, those commonly used are thioglycolic acid, cysteamine, glyceryl monothioglycolate, thiolactic acid and cysteine. Thioglycolic acid is, for example, efficient at reducing the disulfide bonds of keratin at an alkaline pH, such as in the form of ammonium thioglycolate, and is most commonly used in permanent-waving (hair waving). However, it has been found that thioglycolic acid should be used in a sufficiently basic medium (in practice at a pH of between 8.5 and 9.5) if it is desired to obtain curling of sufficient intensity. Besides the drawback of releasing an unpleasant odor requiring the use of more or less efficient fragrances to mask the odors, the use of a thiol at an alkaline pH may also lead to degradation of the fiber such as impairment of the artificial colorations.

Sulfites or bisulfites are mainly used for relaxing. They have drawbacks similar to thiols, with lower efficacy.

Thiols and sulfites (or bisulfites) may also have the drawback of having poor stability in an aqueous solution.

In general, the durability of the reshaping effects obtained with thiols and/or sulfites by reduction of sulfides followed by fixing is considered to be very much inferior to that which may be obtained via the lanthionization technique.

For the second technique, the compositions generally used to perform the lanthionization comprise at least one hydroxide base such as sodium hydroxide, guanidinium hydroxide and lithium hydroxide. These lanthionization active agents, which make it possible to open the disulfide bonds via a beta-elimination mechanism, are generally used in a water-in-oil emulsion at concentrations of between 0.4 and 0.6M, by leaving them to act generally for 10 to 15 minutes at room temperature. Sodium hydroxide is the agent most commonly used. Guanidinium hydroxide is now the preferred compound for many compositions. These two hydroxides, i.e., sodium hydroxide and guanidinium hydroxide, are the two main agents used for relaxing or decurling naturally frizzy/curly hair. They have several advantages over ammonium thioglycolate and sulfites, such as an absence of unpleasant odor, the fact that only one implementation step is required (shorter treatment time), and much greater durability and efficacy of the reshaping of the hair.

However, these hydroxide bases have the major drawback of being caustic. This causticity may affect the scalp by causing irritation that on occasion may be severe. This may be partially overcome by the prior application to the scalp of a greasy protective cream often referred to as “base” or “base cream”, wherein the word “base” as used here does not have the meaning of a basic agent in the chemical sense. When the protective cream is combined with the hydroxide compound in a single composition, this is generally referred to as a “no-base” composition, as opposed to the above name. This “no-base” technology may, for example, be used, in part because it is more convenient.

The causticity of the hydroxides may also affect the state of the hair by, on the one hand, giving it a coarse feel and, on the other hand, making it much more fragile. This fragility may possibly go as far as fraying, breaking or even dissolution of the hair if the treatment is prolonged. In certain cases, hydroxides may also cause decoloration of the natural color of the hair.

Formulations containing sodium hydroxide are generally referred to as “lye relaxers” and those not containing it are known as “no-lye relaxers”.

The main “no-lye” relaxing formulations use guanidinium hydroxide. Since guanidinium hydroxide may be unstable, it is generated extemporaneously by mixing guanidine carbonate and a source of very sparingly soluble hydroxide such as calcium hydroxide. The reaction between these two compounds leads to the formation of guanidinium hydroxide and calcium carbonate, which precipitates in the composition. The presence of this precipitate may make the final rinsing of the hair much more difficult and leave on the hair and the scalp mineral particles that can give it a coarse feel and an unaesthetic appearance resembling dandruff. The recent success of guanidinium hydroxide (“no-lye”) over sodium hydroxide (“lye”) appears to arise from better relaxing efficacy and better skin tolerance. However, these technologies using the hydroxide bases remain very aggressive for the hair and the scalp and may require very strict control of the application time to avoid excessive irritation and impairment of the hair that may go as far as breaking. This aggressiveness arising from the causticity of hydroxides is just reason for these compositions for the lanthionization of the hair not to be used for permanent-waving (hair waving), but instead to be generally reserved for relaxing (hair straightening or hair relaxing).

Furthermore, hydroxides are known to be good agents for hydrolyzing amide functions (cf. for example, March's Advanced Organic Chemistry, 5th Edition, Wiley Interscience, New York, “Hydrolysis of Amides” pages 474 et seq), which thus may lead to breaking of the peptide bonds by direct nucleophilic attack. It is thus possible that the impairments observed in the case of the hair and keratin materials in a broad sense are largely due to partial hydrolysis of the amide bonds of keratin.

There is thus a real need, in relaxing, for compositions that are less aggressive to the hair and the skin.

Various studies have been performed with a view to overcoming at least one of the drawbacks of reducing agents (the first technique) and of hydroxides (the second technique).

Thus, to replace thioglycolic acid, many reducing agents have been proposed, but thioglycolic acid in its ammonium thioglycolate form remains both the reference compound and the compound most widely used in cosmetic formulations, both for shaping and for relaxing and straightening the hair.

To replace sodium hydroxide or guanidinium hydroxide and to improve the skin tolerance, it has been proposed, in U.S. Pat. No. 4,530,830, to use a composition based on quaternary ammonium hydroxides. However, these compositions may not be entirely satisfactory, either in terms of relaxing or in cosmetic terms.

More generally, many publications describe the combined use of hydroxides, acting as lanthionization active agents, with certain additives generally acting to protect the hair.

Specifically, without using novel lanthionization active agents, the improvements proposed relate mainly to the use of additives to reduce the damage caused to the hair by hydroxides. Reference may be made, for example, to:
patent application WO 2002/003937, which describes a composition comprising C3-C5 monosaccharides,
patent application WO 2001/064171, which describes a composition comprising complexing agents,
U.S. Pat. No. 5,641,477, which describes a composition comprising a hydrogenated starch hydrolysate,
patent application WO 02/085317, which describes a composition comprising organic nucleophiles, which react during the second step with the dehydroalanine formed with hydroxides, to give new bridges, and
U.S. Pat. No. 5,679,327, which describes a relaxing composition comprising three active ingredients for relaxing, namely an alkaline hydroxide, an alkaline-earth metal hydroxide, and a nitrogenous organic base, wherein each of the ingredients is present in the composition in a proportion that would be insufficient to effect relaxing if it were used at this same concentration without the other two active ingredients. In other words, the patent describes synergism among the three ingredients, these ingredients together resulting in relaxing of the keratin fibers.

Although all these proposals can lead to more or less marked improvements, they do not make it possible to sufficiently reduce the damage caused by the actual causticity of the hydroxides.

In relation to the use of hydroxides for relaxing, U.S. Pat. No. 4,524,787 has also disclosed a ready-to-use composition made from a virtually anhydrous “activating” part comprising an organic base in a solvent such as a polyhydroxylated alkane, and an aqueous part comprising at least 20% by weight of water. However, this type of the composition may still not be satisfactory, either in terms of quality of relaxing or in terms of mechanical and cosmetic properties of hair that has undergone this treatment.

As indicated previously, the use of reducing agents can lead to mediocre durability of the relaxing or decurling and the use of hydroxides, on account of their causticity, may limit their use in the relaxing field.

After extensive studies, it has now been discovered, entirely surprisingly and unexpectedly, that the first step of the lanthionization process can be performed with tetramethylguanidine. Excellent results in terms of relaxing and cosmetic and mechanical properties of the hair can thus be obtained.

Thus, disclosed herein is a cosmetic composition comprising at least one tetramethylguanidine in a cosmetically acceptable medium, wherein the cosmetically acceptable medium and the concentration of the at least one tetramethylguanidine are chosen such that the at least one tetramethylguanidine is reactive with the cystines of keratin fibers, via a beta-elimination reaction, to produce dehydroalanine and lead to the formation of lanthionine, and thus to relax the keratin fibers in less than 60 minutes.

In one embodiment, the relaxing time is less than 40 minutes such as less than 30 minutes.

In the compositions as disclosed herein intended for a hair relaxing, decurling or straightening process, the at least one tetramethylguanidine is, for example, present in a molar concentration ranging from 0.1 M to 2 M, which corresponds to a concentration ranging from 1.15% to 23% by weight relative to the total weight of the composition, such as in a molar concentration ranging from 0.2 M to 1 M, which corresponds to a concentration ranging from 2.3% to 11.5% by weight relative to the total weight of the composition.

The pH of the composition as disclosed herein is, for example, ranging from 9.6 to 14 such as from 11 to 13.

In one embodiment, in the compositions as disclosed herein, the at least one tetramethylguanidine is the only relaxing active agent.

The compositions as disclosed herein may also comprise at least one known reducing agent chosen, for example, from thioglycolic acid and thiolactic acid and ester and amide derivatives thereof, such as glyceryl monothioglycolate, cysteamine and its C1-C4 acyl derivatives, such as N-acetylcysteamine and N-propionylcysteamine, cysteine, N-acetylcysteine, thiomalic acid, pantethine, 2,3-dimercaptosuccinic acid, sulfites and bisulfites of an alkali metal or an alkaline-earth metal, the N-(mercaptoalkyl)-ω-hydroxyalkylamides described in patent application EP-A-354 835, the N-mono- or N,N-dialkylmercapto-4-butyramides described in patent application EP-A-368 763, the aminomercaptoalkylamides described in patent application EP-A432 000, the N-(mercaptoalkyl)succinamic acid and N-(mercaptoalkyl)succinimide derivatives described in patent application EP-A-465 342, the alkylamino mercaptoalkylamides described in patent application EP-A-514 282, the azeotropic mixture of 2-hydroxypropyl thioglycolate and of (2-hydroxy-1-methyl)ethyl thioglycolate described in patent application FR-A-2 679 448, the mercaptoalkylaminoamides described in patent application FR-A-2 692 481, the N-mercaptoalkylalkanediamines described in patent application EP-A-653 202 and the formamidinesulfinic acid derivatives described in patent application PCT/US 01/43124.

When the compositions as disclosed herein comprise at least one reducing agent, this at least one reducing agent is, for example, present in a maximum concentration of 20% by weight such as from 0.1% to 10% by weight relative to the total weight of the composition.

The compositions as disclosed herein may also comprise at least one known hydroxide chosen, for example, from alkali metal hydroxides, alkaline-earth metal hydroxides, transition metal hydroxides, and organic hydroxides, such as sodium hydroxide, potassium hydroxide, lithium hydroxide, rubidium hydroxide, caesium hydroxide, francium hydroxide, beryllium hydroxide, magnesium hydroxide, calcium hydroxide, strontium hydroxide, barium hydroxide, molybdenum hydroxide, manganese hydroxide, zinc hydroxide, cobalt hydroxide, cadmium hydroxide, cerium hydroxide, lanthanum hydroxide, actinium hydroxide, thorium hydroxide, aluminium hydroxide, guanidinium hydroxide and quaternary ammonium hydroxides.

When the compositions as disclosed herein comprise at least one hydroxide, this at least one hydroxide is, for example, present in a concentration ranging from 0.01% to 3.5% by weight such as from 0.05% to 1.5% by weight relative to the total weight of the composition.

In one embodiment, the composition as disclosed herein comprises 0% of a hydroxide base chosen, for example, from alkali metal hydroxides, alkaline-earth metal hydroxides, transition metal hydroxides, and organic hydroxides.

In one embodiment, the composition as disclosed herein comprises from 0 to 50%, such as from 0 to 30% and further such as from 0 to 20% by weight of water relative to the total weight of the composition.

In another embodiment, the composition disclosed herein also comprises at least one surfactant chosen from nonionic, anionic, cationic and amphoteric surfactants, and among these surfactants, mention may be made, for example, of alkyl sulfates, alkylbenzene sulfates, alkyl ether sulfates, alkyl sulfonates, quaternary ammonium salts, alkylbetaines, oxyethylenated alkylphenols, fatty acid alkanolamides, oxyethylenated fatty acid esters and other nonionic surfactants of the hydroxypropyl ether type.

When the composition disclosed herein comprises at least one surfactant, this at least one surfactant is, for example, present in a maximum concentration of 30% by weight such as from 0.5% to 10% by weight relative to the total weight of the composition.

In order to improve the cosmetic properties of the hair or to attenuate or avoid its degradation, the composition as disclosed herein may also comprise at least one treating agent chosen from treating agents of cationic, anionic, nonionic and amphoteric nature.

Among the treating agents, mention may be made, for example, of those described in French patent nos. 2 598 613 and 2 470 596. It is also possible to use, as treating agents, volatile or non-volatile, linear or cyclic silicones and mixtures thereof, polydimethylsiloxanes, quaternized polyorganosiloxanes such as those described in French patent application no. 2 535 730, polyorganosiloxanes comprising aminoalkyl groups modified with alkoxycarbonylalkyl groups, such as those described in U.S. Pat. No. 4,749,732, polyorganosiloxanes such as polyoxyalkyl polydimethylsiloxane copolymer of the Dimethicone Copolyol type, a polydimethylsiloxane comprising stearoxy end groups (stearoxy dimethicone), a dialkylammonium acetate polydimethylsiloxane copolymer and a polydimethylsiloxane polyalkylbetaine copolymer described in British patent no. 2 197 352, polysiloxanes organomodified with mercapto or mercaptoalkyl groups, such as those described in French patent no. 1 530 369 and in European patent application 295 780, and silanes such as stearoxytrimethylsilane.

The composition as disclosed herein may also comprise at least one other treating ingredient such as cationic polymers, for instance those used in the compositions of French patent nos. 79/32078 (2 472 382) and 80/26421 (2 495 931) and cationic polymers of the ionene type, such as those used in the compositions of Luxembourg patent no. 83703, basic amino acids (such as lysine and arginine), acidic amino acids (such as glutamic acid and aspartic acid), peptides and derivatives thereof, protein hydrolysates, waxes, swelling agents and penetrating agents and agents for reinforcing the efficacy of the reducing agent, such as the SiO₂/PDMS (polydimethylsiloxane) mixture, dimethyl-isosorbitol, urea and its derivatives, pyrrolidone, N-alkylpyrrolidones, thiamorpholinone, alkylene glycol and dialkylene glycol alkyl ethers, for instance propylene glycol monomethyl ether, dipropylene glycol monomethyl ether, ethylene glycol monoethyl ether and diethylene glycol monoethyl ether, 1,2-propanediol, 1,3-propanediol, glycerol, 2-imidazolidinone, and also other compounds such as fatty alcohols, lanolin derivatives, active ingredients such as pantothenic acid, agents for preventing hair loss, antidandruff agents, thickeners, suspending agents, sequestering or complexing agents, opacifiers, sunscreens, fragrances and preserving agents.

As used herein, the term “cosmetically acceptable medium” is known to one of ordinary skill in the art, and may comprise, for example, water and/or at least one organic solvent.

In one embodiment, in the compositions as disclosed herein, the at least one tetramethylguanidine relaxes keratin fibers without being placed in contact beforehand with an organic solvent.

The composition disclosed herein is, for example, in the form of a thickened cream so as to hold the hair as stiff as possible. These creams are made in the form of “heavy” emulsions, for example, based on glyceryl stearate, glycol stearate, self-emulsifying waxes or fatty alcohols.

Liquids or gels containing thickeners, such as carboxyvinyl polymers or copolymers that “stick” the hairs together and hold them in a smooth position during the leave-in time, may also be used.

The composition as disclosed herein may also comprise at least one adjuvant chosen, for example, from silicones in soluble, dispersed and microdispersed forms, nonionic, anionic, cationic and amphoteric surfactants, ceramides, glycoceramides and pseudoceramides, vitamins and provitamins including panthenol, plant, animal, mineral and synthetic oils, waxes other than ceramides, glycoceramides and pseudoceramides, water-soluble and liposoluble, silicone-based and non-silicone-based sunscreens, nacreous agents and opacifiers, sequestering agents, plasticizers, solubilizers, acidifying agents, mineral and organic thickeners, antioxidants, hydroxy acids, penetrating agents, fragrances and preserving agents. Among the solubilizers, mention may be made, for example, of lower alcohols, for instance ethanol, propanol and isopropanol.

This disclosure also relates to a kit comprising at least two compartments, wherein one of the compartments comprises a composition (i) comprising at least one tetramethylguanidine in a cosmetically acceptable medium, wherein the at least one tetramethylguanidine is capable of reacting with the cystines of keratin fibers, via a beta-elimination reaction producing dehydroalanine and leading to the formation of lanthionine, to relax the keratin fibers in less than 60 minutes.

In one embodiment, the kit as disclosed herein also comprises an additional composition (ii) for caring for, conditioning, making up, removing makeup from, protecting, cleansing and/or washing keratin fibers.

The compositions of the kits as disclosed herein are packaged in separate compartments, containers or devices, optionally accompanied by suitable, identical or different application tools, such as fine brushes, coarse brushes and sponges.

This disclosure also relates to a process for relaxing a keratin material using a cosmetic composition comprising at least one tetramethylguanidine in a cosmetically acceptable medium, wherein the cosmetically acceptable medium and the concentration of the at least one tetramethylguanidine are chosen such that the at least one tetramethylguanidine is reactive with the cystines of the keratin material, via a beta-elimination reaction to produce dehydroalanine and to lead to the formation of lanthionine, to relax the keratin material in less than 60 minutes.

In one embodiment, in the process as disclosed herein, the relaxing time is less than 40 minutes such as less than 30 minutes.

In the hair relaxing, decurling or straightening process as disclosed herein, the composition as disclosed herein is applied to the hair and the hair is then subject to mechanical reshaping allowing it to be given a new shape, via an operation of smoothing-out the hair via a large-toothed comb, with the back of a comb, or by hand. After a leave-in time ranging from 5 to 60 minutes such as from 5 to 40 minutes, the hair is smoothed out again and is then rinsed thoroughly.

After applying the composition as disclosed herein, the head of hair may, for example, be subject to a heat treatment by heating to a temperature ranging from 30 to 60° C. In practice, this operation may be performed using a hairstyling hood, a hair drier, an infrared ray dispenser and/or other standard heating devices.

It is possible to use a hot iron at a temperature ranging from 60 to 220° C. such as from 120 to 200° C. as a method of both heating and smoothing out the hair.

Further disclosed herein is the use of at least one tetramethylguanidine as an active agent for relaxing keratin fibers.

The present disclosure also relates to an active agent for relaxing keratin fibers, by a beta-elimination reaction producing dehydroalanine and leading to the formation of lanthionine, comprising at least one tetramethylguanidine.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The invention may be understood more clearly with the aid of the non-limiting examples that follow.

### EXAMPLE 1

A simplified relaxing composition was prepared, comprising tetramethylguanidine at a concentration of 0.8 M in water, as relaxing active agent. The pH of the composition was 13.3. This composition was applied to naturally frizzy/curly African hair for 15 minutes at a temperature of 30° C. The hair was relaxed, easy to comb, and felt soft.

### EXAMPLE 2

A simplified relaxing composition was prepared, comprising tetramethylguanidine at a concentration of 0.5 M in water, as relaxing active agent. The pH of the composition was 13.2. This composition was applied to naturally frizzy/curly African hair for 25 minutes at a temperature of 30° C. The hair was relaxed, easy to comb, and felt soft.

1. A cosmetic composition comprising at least one tetramethylguanidine in a cosmetically acceptable medium, wherein the cosmetically acceptable medium and the concentration of the at least one tetramethylguanidine are chosen such that the at least one tetramethylguanidine is reactive with the cystines of keratin fibers, via a beta-elimination reaction to produce dehydroalanine and lead to the formation of lanthionine, to relax the keratin fibers in less than 60 minutes.

2. The composition according to claim 1, wherein the relaxing time is less than 40 minutes.

3. The composition according to claim 2, wherein the relaxing time is less than 30 minutes.

4. The composition according to claim 1, wherein the at least one tetramethyl-guanidine is present in a molar concentration ranging from 0.1 M to 2 M.

5. The composition according to claim 4, wherein the at least one tetramethyl-guanidine is present in a molar concentration ranging from 0.2 M to 1 M.

6. The composition according to claim 1, wherein the pH of the composition ranges from 9.6 to 14.

7. The composition according to claim 6, wherein the pH of the composition ranges from 11 to 13.

8. The composition according to claim 1, wherein the composition comprises 0% of a hydroxide base.

9. The composition according to claim 1, wherein the composition comprises from 0 to 50% by weight of water, relative to the total weight of the composition.

10. The composition according to claim 9, wherein the composition comprises from 0 to 30% by weight of water, relative to the total weight of the composition.

11. The composition according to claim 10, wherein the composition comprises from 0 to 20% by weight of water, relative to the total weight of the composition.

12. The composition according to claim 1, further comprising at least one adjuvant chosen from silicones in soluble, dispersed and microdispersed forms, nonionic, anionic, cationic and amphoteric surfactants, ceramides, glycoceramides and pseudoceramides, vitamins and provitamins, plant, animal, mineral and synthetic oils, waxes other than ceramides, glycoceramides and pseudoceramides, water-soluble and liposoluble, silicone-based and non-silicone-based sunscreens, nacreous agents and opacifiers, sequestering agents, plasticizers, solubilizers, acidifying agents, mineral and organic thickeners, antioxidants, hydroxy acids, penetrating agents, fragrances and preserving agents.

13. The composition according to claim 1, wherein the composition is in a form of a cream, a liquid, and/or a gel comprising at least one thickener.

14. A kit comprising at least two compartments, wherein one of the compartments comprises a composition (i) comprising at least one tetramethylguanidine in a cosmetically acceptable medium, wherein the at least one tetramethylguanidine is reactive with the cystines of keratin fibers, via a beta-elimination reaction to produce dehydroalanine and to lead to the formation of lanthionine, to relax the keratin fibers in less than 60 minutes.

15. The kit according to claim 14, further comprises an additional composition (ii) for caring for, conditioning, making up, removing makeup from, protecting, cleansing and/or washing keratin fibers.

16. A process for relaxing keratin fibers comprising
applying to the keratin fibers a cosmetic composition comprising at least one tetramethylguanidine in a cosmetically acceptable medium, wherein the cosmetically acceptable medium and the concentration of the at least one tetramethylguanidine are chosen such that the at least one tetramethylguanidine reacts with the cystines of the keratin material, via a beta-elimination reaction producing dehydroalanine and leading to the formation of lanthionine, to relax the keratin fibers in less than 60 minutes.

17. The process according to claim 16, wherein the keratin fibers are head hair.

18. The process according to claim 16, further comprising, after applying the composition, heating the keratin fibers to a temperature ranging from 30 to 60° C.

19. The process according to claim 16, further comprising heating and smoothing out the keratin fibers with a hot iron at a temperature ranging from 60 to 220° C.

20. The process according to claim 19, wherein the hot iron is at a temperature ranging from 120 to 200° C.

21. The process according to claim 16, wherein the relaxing time is less than 40 minutes.

22. The process according to claim 21, wherein the relaxing time is less than 30 minutes.

23. An active agent for relaxing keratin fibers, comprising at least one tetramethylguanidine, wherein the at least one tetramethylguanidine is reactive with the cystines of the keratin fibers via a beta-elimination reaction producing dehydroalanine and leading to the formation of lanthionine, to relax the keratin fibers.
